(19) United States
(12) Patent Application Publication
(10) Pub. No.: US 2024/0010678 A1
(43) Pub. Date: Jan. 11, 2024

(54) METHOD OF TREATMENT OF IRRITABLE BOWEL SYNDROME

(71) Applicant: THE JOHNS HOPKINS UNIVERSITY, Baltimore, MD (US)

(72) Inventors: Pankaj J. Pasricha, Ellicott City, MD (US); Phillip Phan, Baltimore (MD); Lian Sheng Liu, Baltimore, MD (US); Qian Li, Baltimore, MD (US)

(21) Appl. No.: 18/025,367

(22) PCT Filed: Sep. 8, 2021
(86) PCT No.: PCT/US21/49459

§ 371 Date: Mar. 8, 2023

Related U.S. Application Data
(60) Provisional application No. 63/075,794, filed on Sep. 8, 2020.

(51) Int. Cl. C07K 7/06; A61K 31/437; A61K 45/06; A61P 1/00

(52) U.S. Cl. CPC C07K 7/06; A61K 31/437; A61K 45/06; A61P 1/00

(57) ABSTRACT

Methods of treating irritable bowel syndrome (IBS) include the administration of an effective amount of an anti-microbial decapeptide NH₂-Lys-Lys-Val-Val-Phe-Trp-Val-Lys-Phe-Lys-CO NH₂ (SEQ ID NO 1) (KSL-W).

The present application claims the benefit of priority of U.S. provisional application No. 63/075,794, filed Sep. 8, 2020, which is incorporated by reference herein in its entirety.

### FIELD OF THE DISCLOSURE

Compositions which include antimicrobial peptides are provided for use in treating intestinal diseases.

### BACKGROUND

Disorders involving abnormal functioning of the gastrointestinal tract afflict large segments the world's population. The most prevalent of the functional disorder in the absence of structural abnormalities is irritable bowel syndrome (IBS). The most common inflammatory gastrointestinal diseases are inflammatory bowel disease (IBD, which includes Crohn's disease, ulcerative colitis and indeterminate colitis)and gastritis. Irritable Bowel Syndrome (IBS) is a chronic, episodic gastrointestinal disorder that is characterized by abdominal pain and altered bowel habit. IBS prevalence is estimated to be 10-15% in Western countries comprising 25 to 50 percent of all referrals to gastroenterologists. The gastrointestinal tract harbors a complex and diverse microbial community, which plays important roles in host nutrition, immune function, health and disease, and it is hypothesized the IBS disease phenotype is associated with a change in colonic microbiota and/or host factors such as mucosal function and immunity.

Current treatments of IBS with probiotics and antibiotics are not uniformly effective (Moayyedi P, Ford A C, Talley N J, et al., “The efficacy of probiotics in the treatment of irritable bowel syndrome: a systematic review.” Gut 2010;59:325-332; Jailwala, Jeegar, Thomas F. Imperiale, and Kurt Kroenke. “Pharmacologic treatment of the irritable bowel syndrome: a systematic review of randomized, controlled trials.” Annals of Internal Medicine 133.2 (2000): 136-147; Ford, Alexander C., et al. “Effect of fibre, antispasmodics, and peppermint oil in the treatment of irritable bowel syndrome: systematic review and meta-analysis.” British Medical Journal 337 (2008): a2313).

### SUMMARY

There is a high frequency of incomplete or absent response of IBS and other gastrointestinal to current medical therapies. Thus there is an urgent and unmet need for improved methods, compositions, etc. that can treat or ameliorate one or more symptoms associated with these diseases.

Accordingly, a method of treating inflammatory gastrointestinal diseases, such as for example, IBS, comprises administering to a subject in need of such treatment, a pharmaceutical composition comprising a therapeutically effective amount of a peptide comprising at least 90% sequence identity to NH₂-Lys-Lys-Val-Val-Phe-Trp-Val-Lys-Phe-Lys-CO NH₂ (SEQ ID NO 1) derivatives or analogs thereof. In certain embodiments, the peptide comprises SEQ ID NO: 1.

In certain embodiments, a method of treating irritable bowel syndrome (IBS), comprising: administering to a subject in need of such treatment, a pharmaceutical composition comprising a therapeutically effective amount of one or more antimicrobial peptides comprising SEQ ID NOS: 1-4 or combinations thereof.

In certain embodiments, a method of treating a bowel disease comprises: administering to a subject in need of such treatment, a pharmaceutical composition comprising a therapeutically effective amount of one or more antimicrobial peptides comprising one or more of SEQ ID NOS: 1-4 or combinations thereof. A bowel disease comprises any one or more of: irritable bowel syndrome (IBS), diarrhea-predominant Irritable Bowel Syndrome (dIBS), inflammatory bowel disease (IBD), Crohn's disease, post-surgical pouchitis, diverticulitis, traveler's diarrhea, ulcerative colitis, enteritis, small intestinal bacterial overgrowth, chronic pancreatitis, pancreatic insufficiency, colitis, diverticular disease, colorectal cancer, lymphocytic colitis, collagenous colitis, diversion colitis, endometriosis, caustic enema-induced colitis, drug-induced ischemic colitis, and/or hepatic encephalopathy.

In certain embodiments, the peptides are administered to a subject orally or via an enteral route. In certain embodiments, the peptides are administered to a subject via a rectal route.

In certain embodiments, the method further comprises administering to the subject, one or more prebiotics, probiotics, antibiotics, anti-inflammatory agents or combinations thereof. In certain embodiments, the one or more antibiotics comprise: penicillins, tetracyclines, cephalosporins, quinolones, lincomycins, macrolides, sulfonamides, glycopeptides, aminoglycosides, carbapenems or combinations thereof.

In certain embodiments, a pharmaceutical compositions comprises a therapeutically effective amount of one or more peptides comprising SEQ ID NOS: 1-4, derivatives or analogs thereof.

In certain embodiments, a pharmaceutical compositions comprises a therapeutically effective amount of one or more peptides comprising SEQ ID NOS: 1-4, derivatives or analogs thereof, one or more prebiotics, probiotics, antibiotics, anti-inflammatory agents or combinations thereof. In certain embodiments, the one or more antibiotics comprise: penicillins, tetracyclines, cephalosporins, quinolones, lincomycins, macrolides, sulfonamides, glycopeptides, aminoglycosides, carbapenems or combinations thereof.

In certain embodiments the antimicrobial peptide is SEQ ID NO: 1, derivatives or analogs thereof. In certain embodiments, the antimicrobial peptides are pegylated.

In certain embodiments, the antibiotic is rifaximin.

In certain embodiments, a therapeutically effective dose of the one or more antimicrobial peptides comprises a range from about 0.1 mg up to 500 mg. In certain embodiments, a therapeutically effective dose of the one or more antimicrobial peptides comprises a range from about 0.2 mg to about 450 mg, or from about 0.3 mg to about 400 mg, or from about 0.3 mg to about 375 mg, or from about 0.4 mg to about 350 mg, or from about 0.5 mg to about 350 mg, or from about 0.6 mg to about 325 mg, or from about 0.7 mg to about 300 mg, or from about 0.7 mg to about 300 mg, or from about 0.8 mg to about 275 mg, or from about 0.9 mg to about 250 mg, or from about 1 mg to about 245 mg, or from about 1 mg to about 240 mg, or from about 1 mg to about 235 mg, or from about 1 mg to about 230 mg, or from about 1.0 mg to about 225 mg, or from about 1 mg to about 220 mg, or from about 1 mg to about 210 mg, or from about 1 mg to about 200 mg, or from about 1 mg to about 175 mg, or from about 1 mg to about 150 mg, or from about 1 mg to about 145 mg or from about 1 mg to about 140 mg, or from about 1 mg to about 135 mg, or from about 1 mg to about 130 mg, or from about 1 mg to about 125 mg, or from about 1 mg to about 120 mg, or from about 1 mg to about 115 mg, or from about 1 mg to about 110 mg, or from about 1 mg to about 100 mg.

In certain embodiments, a therapeutically effective dose of the one or more antimicrobial peptides comprises a range from about 2 mg up to 100 mg.

In certain embodiments, the pharmaceutical compositions comprising the one or more antimicrobial peptides, e.g. SEQ ID NOS: 1-4, are administered once a day, twice a day, three times a day or as needed.

Other aspects are discussed infra.

Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “effective dose” or “effective dosage” is defined as an amount sufficient to achieve or at least partially achieve the desired effect.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used in this specification and the appended claims, the term “or” is generally employed in its sense including “and/or” unless the content clearly dictates otherwise.

The term “osmolarity” refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na⁺ and Cl⁻ in solution and thus provides 2 moles of dissolved components per 1 mole of dissolved NaCl in solution. Physiological osmolarity is typically in the range of about 280 to about 310 mOsm/L.

The term “patient” includes human and other mammalian subjects that receive either prophylactic or therapeutic treatment.

The term “therapeutically effective amount” refers to an amount of a therapeutic or prophylactic agent, such as a biologic agent, that, when incorporated into and/or onto the self-assembled gel composition, produces some desired effect at a reasonable benefit/risk ratio applicable to any treatment. The effective amount may vary depending on such factors as the disease, disorder or condition being treated, the particular formulation being administered, the size of the subject, or the severity of the disease, disorder or condition.

The term “tonicity” refers to the osmotic pressure gradient resulting from the separation of two solutions by a semi-permeable membrane. In particular, tonicity is used to describe the osmotic pressure created across a cell membrane when a cell is exposed to an external solution. Solutes that readily cross the cellular membrane contribute minimally to the final osmotic pressure gradient. In contrast, those dissolved species that do not cross the cell membrane, “impermeable solutes”, will contribute to osmotic pressure differences and thus tonicity. The term “hypertonic”, as generally used herein, refers to a solution with a higher concentration of impermeable solutes than is present on the inside of the cell. When a cell is immersed into a hypertonic solution, water will flow out of the cell, concentrating the impermeable solutes inside the cell until it becomes equal to the concentration of impermeable solutes outside the cell. The term “hypotonic” refers to a solution with a lower concentration of impermeable solutes than is present inside of the cell. When a cell is immersed into a hypotonic solution, water will flow into the cell, diluting the concentration of impermeable solutes inside the cell until it becomes equal to the concentration of impermeable solutes outside the cell. The term “isotonic” refers to a solution wherein the osmotic pressure gradient across the cell membrane is essentially balanced and no water flows into or out of the cell. The same meanings for tonicity apply for water flow through intestinal epithelia; hypertonic solutions cause water to flow into the lumen, whereas hypotonic solutions cause water to flow out of the lumen. Tonicity depends on the permeability properties of the cell or epithelium to different solutes, whereas osmolarity depends only on the total concentration of all solutes.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder or condition includes ameliorating at least one symptom of the particular disease, disorder or condition, even if the underlying pathophysiology is not affected, such as treating the pain of a subject by administration of an analgesic agent even though such agent does not treat the cause of the pain.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder or condition includes ameliorating at least one symptom of the particular disease, disorder or condition, even if the underlying pathophysiology is not affected, such as treating the pain of a subject by administration of an analgesic agent even though such agent does not treat the cause of the pain.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE FIGURES

FIG. 1 is a graph demonstrating the effects of intracolonic KSL-W at two different concentrations on pain behavior in a mouse model of IBS. Behavioral responses are quantified along the Y-axis (VMR=visceromotor reflex, a measure of pain behavior in response to visceral stimulation, assessed by electromyographic activity recorded from abdominal wall muscle). The numbers on the x-axis represent the different pressures generated by an intracolonic balloon using a graded distention protocol to provide noxious stimulation to the colon.

FIG. 2 is a graph showing the effect of rifaximin in colorectal distention (CRD).

### DETAILED DESCRIPTION

Antimicrobial peptides isolated from a variety of natural sources have received attention because of their selectivity for prokaryotes and promise of minimizing microbial resistance. A novel antimicrobial decapeptide (KSL) and some of its analogs have been shown to possess a broad range of antibacterial activity as well as inhibit the growth of oral bacterial strains associated with caries development and plaque formation (U.S. Pat. Nos. 7,494,980 and 8,778,889 which are incorporated by reference herein in their entirety).

The KSL peptide was shown to be effective in preventing the formation of biofilms that mimic supragingival or cariogenic plaque, in inhibiting the growth of various oral microorganisms and in preventing the formation of biofilms. Examples of such microorganisms, included Fusobacterium nucleatum, Peptostreptococcus micros, Eubacterium timidum, Eubacterium brachy, Lactobacillus spp., Actinomyces naeslundii, Pseudomonas anaerobius, Eubacterium sp. strain D8, Prevotella intermedia, Fusobacterium sp., Selenomonas sputigena, Eubacterium sp. strain D6, Bacteroides pneumosintes, Haemophilus aphrophilus, Actinomyces israelli, S. mutans, S. gordonii, S. sanguis, S. oralis, S. sobrinus, S. salivarius, S. mitis, L. salivarius, Porphyromonas gingivalis, Tanerella forsythensis, Dialister pneumosintes, Veillonella parvula, L. acidophilus, Staphylococcus aureus ATCC 6538, Methicillin-resistant Staphylococcus aureus, Staphylococcus epidermidis ATCC 12228, Micrococcus luteus ATCC 9341, Mycobacterium smegmatis ATCC 607, Corynebacterium diphtheriae ATCC 8024, Escherichia coli ATCC 2592, Pseudomonas aeruginosa ATCC 9027, Proteus vulgaris ATCC 6380, Shigella flexneri ATCC 203, or Candida albicans ATCC 36232.

The human gastrointestinal (GI) tract represents one of the largest interfaces (250-400 m²) between the host, environmental factors and antigens in the human body. In an average life time, around 60 tons of food pass through the human GI tract, along with an abundance of microorganisms from the environment which impose a huge threat on gut integrity (Bengmark S. Ecological control of the gastrointestinal tract. The role of probiotic flora. Gut. 1998 January; 42(1):2-7). The collection of bacteria, archaea and eukarya colonizing the GI tract is termed the ‘gut microbiota’ and has co-evolved with the host over thousands of years to form an intricate and mutually beneficial relationship (Backhed F, et al., Science. 2005 Mar. 25; 307(5717):1915-20. Neish A S. Gastroenterology. 2009 January; 136(1):65-80). The number of microorganisms inhabiting the GI tract has been estimated to exceed 10¹⁴, which encompasses ˜10 times more bacterial cells than the number of human cells and over 100 times the amount of genomic content (microbiome) as the human genome (Bäckhed F, et al., Science. 2005 Mar. 25; 307(5717):1915-20. Gill S R, et al., Science. 2006 Jun. 2; 312(5778):1355-9. Thursby, E., & Juge, N. (2017). Introduction to the human gut microbiota. The Biochemical journal, 474(11), 1823-1836. https://doi.org/10.1042/BCJ20160510).

Many diseases and conditions, or stages thereof, are believed to be associated with perturbations in the microbiota of the GI tract, or regions thereof. In some instances the disease or condition may be caused by, or is exacerbated by, the shift in the profile of the microbiota of the GI tract, or regions thereof (i.e. the relative amounts of constituent microbes and the diversity of those microbes). In other instances the disease or condition causes, or by some mechanism results in, the display of a profile of the microbiota of the GI tract that differs from the normal state. In some contexts this may even be an adaptive response attempting to address the pathological phenotype of the disease or condition. Indeed, it has been suggested that disruption of the gut microbiota (or dysbiosis) can be significant with respect to pathological intestinal conditions such as obesity (Ley R E, et al., Nature. 2006 Dec. 21; 444(7122):1022-3.) [Ley et al. 2006b; Zhang H, et al., Proc Natl Acad Sci USA. 2009 Feb. 17; 106(7):2365-70) and malnutrition (Kau A L, et al., Nature. 2011 Jun. 15; 474(7351):327-36), systematic diseases such as diabetes [Qin J, et al. Nature. 2012 Oct. 4; 490(7418):55-60) and chronic inflammatory diseases such as inflammatory bowel disease (IBD), encompassing ulcerative colitis (UC) and Crohn's disease (CD) [(Frank D N, et al. Proc Natl Acad Sci USA. 2007 Aug. 21; 104(34):13780-5. Guinane, Caitriona M, and Paul D Cotter. “Role of the gut microbiota in health and chronic gastrointestinal disease: understanding a hidden metabolic organ.” Therapeutic advances in gastroenterology vol. 6,4 (2013): 295-308. doi:10.1177/1756283X13482996).).

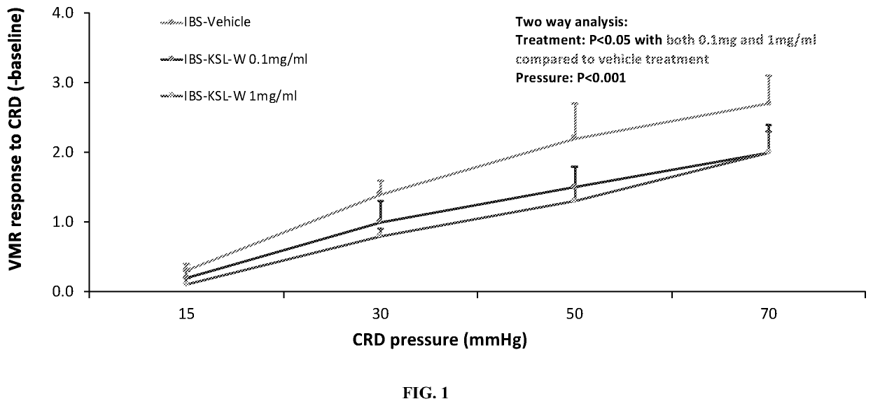

The present invention is based, in part, on the discovery that an antimicrobial peptide used to inhibit the growth of oral bacterial strains associated with caries development and plaque formation is effective in alleviating symptoms and treating IBS and other gastrointestinal diseases or disorders. The KSL-W, an antimicrobial peptide was found to be selective against pathogenic bacteria but not beneficial bacteria, and was found to resets the microbial balance in the colon (FIG. 1). The results obtained revealed a significantly increased compositional difference in the microbial communities in “IBS” animals as compared with controls. Even more striking was the dramatic change in the ratio of Firmicutes relative to Bacteroidetes, where “IBS” animals were enriched more with Bacteroidetes and also contained a different composition of species within this phylum.

The present invention, is also based in part, on the discovery that delivery of an antimicrobial peptide that was found to be degraded rapidly in the stomach, could be formulated for a colonic indication. Formulations are also provided herein.

Accordingly, in certain embodiments, a method of treating inflammatory gastrointestinal diseases, such as for example, IBS, comprises administering to a subject in need of such treatment, a pharmaceutical composition comprising a therapeutically effective amount of a peptide formulated for colonic indication. In certain embodiments, the antimicrobial peptide comprises the amino acid sequences set forth as SEQ ID NO: 1. The antimicrobial peptide, termed herein “KSL-W” (SEQ ID NO: 1) is an L-tryptophan analog of NH₂-Lys-Lys-Val-Val-Phe-Lys-Val-Lys-Phe-Lys-CO NH₂ (SEQ ID NO:4) wherein the L-lysine residue at the number six position is replaced with L-tryptophan. The sequence is: NH₂-Lys-Lys-Val-Val-Phe-Trp-Val-Lys-Phe-Lys-CO NH₂ (SEQ ID NO 1). In some embodiments, the antimicrobial peptides have at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, and any number or range in between, identity to SEQ ID NO:1.

Other analogs of SEQ ID NO: 4, termed herein as KSL, include SEQ ID NOS: 2 and 3.

SEQ ID NO: 2, termed herein KSL-M, is an analog of KSL wherein the L-lysine residue at the number six position is replaced with an α-methylated L-lysine. The sequence is: NH₂-Lys-Lys-Val-Val-Phe-Lys(me)-Val-Lys-Phe-Lys-CO NH₂ (SEQ ID NO 2). In some embodiments, the antimicrobial peptides have at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, and any number or range in between, identity to SEQ ID NO: 2.

SEQ ID NO: 3, termed herein H1-2, is a double helical hybrid molecule consisting of KSL-W and an analog of the N-terminal sequence derived from a salivary protein, statherin. The sequence is: NH₂-Asp-Asp-Asp-Glu-Glu-Lys-Phe-Leu-Arg-Arg-Ile-Gly-Arg-Tyr-Gly-Lys-Lys-Val-Val-Phe-Trp-Val-Lys-Phe-Lys-CO NH₂ (SEQ ID NO 3). In some embodiments, the antimicrobial peptides have at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, and any number or range in between, identity to SEQ ID NO:3.

SEQ ID NO: 4, termed herein KSL, comprises the peptide sequence: NH₂-Lys-Lys-Val-Val-Phe-Lys-Val-Lys-Phe-Lys-CO NH₂ (SEQ ID NO:4). In some embodiments, the antimicrobial peptides have at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, and any number or range in between, identity to SEQ ID NO:4.

Accordingly, in certain embodiments, a method of treating IBD or other gastrointestinal diseases comprises administering a pharmaceutical composition comprising a therapeutically effective amount of one or more peptides comprising SEQ ID NO: 1, SEQ ID NO: 2, SEQ ID NO: 3, SEQ ID NO: 4 or any combinations thereof.

In certain embodiments, the pharmaceutical compositions are administered to a subject for preventing an acute phase of the inflammatory bowel disease. In certain embodiments, the pharmaceutical compositions are administered to a subject for preventing flare-ups wherein the subject having inflammatory bowel disease is in remission.

In certain embodiments, the pharmaceutical composition comprises SEQ ID NO: 1.

In certain embodiments, the pharmaceutical composition comprises SEQ ID NO: 2.

In certain embodiments, the pharmaceutical composition comprises SEQ ID NO: 3.

In certain embodiments, the pharmaceutical composition comprises SEQ ID NO: 4.

A description of SEQ ID NOS: 1-4 is provided in U.S. Pat. Nos. 7,494,980 and 8,778,889 which are incorporated by reference herein in their entirety.

In certain embodiments, the pharmaceutical composition further comprises one or more derivatives of any one of SEQ ID NOS: 1-4. A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

In certain embodiments, a pharmaceutical compositions comprises a therapeutically effective amount of one or more peptides comprising SEQ ID NOS: 1-4, derivatives or analogs thereof, one or more prebiotics, probiotics, antibiotics, anti-inflammatory agents, secondary agents or combinations thereof. In certain embodiments, the one or more antibiotics comprise: penicillins, tetracyclines, cephalosporins, quinolones, lincomycins, macrolides, sulfonamides, glycopeptides, aminoglycosides, carbapenems or combinations thereof.

The secondary agents may have activity as anti-inflammatory agents, non-anti-inflammatory agents, steroids, anesthetics such as lidocaine or benzocaine, analgesics, anti-pyretic agents, anti-infectious agents such as antibacterial agents, anti-protozoal agents antifungal agents, and antiviral agents, immunosuppressants, chemotherapeutics, growth factors, cytokines, immunomodulatory molecules, anti-neoplastic agents, erectile dysfunction improvement agents, anxiolytic agents, sedatives, hypnotics, neuroleptics, blockers, diuretics, opioid analgesics, anti-urinary incontinence agents, nutritional oils, anti-benign prostate hypertrophy agents, essential fatty acids, non-essential fatty acids, vitamins, minerals, or combinations thereof.

### Formulations for Administration

Oral Administration: In certain embodiments, the pharmaceutical compositions for use in the treatment of inflammatory bowel diseases is administered orally. Oral administration in context of the present disclosure means the introduction of the composition into gastrointestinal tract via the mouth. In certain embodiments, the composition is a solid dosage form, such as in the form of a pellet, granule, micro particle, nano particle, mini tablet, capsule or tablet coated with a coating material that prevents the release of the composition before the ileocolonic region of the intestine. The ileocolonic region is the region of the gastrointestinal tract where the small intestine merges with the large intestine, i.e. the terminal ileum.

The compositions can be encapsulated, e.g. nanoparticles, in a tablet form or encapsulated particles in a suspension. The compositions can be coated so that the pharmaceutical agents are released in the intestines and not in the stomach. Coating materials for the targeted release of a composition in the large intestinal lumen are known in the art. They can be subdivided into coating materials that disintegrate above a specific pH, coating materials that disintegrate after a specific residence time in the gastrointestinal tract and coating materials that disintegrate due enzymatic triggers specific to the microflora of the large intestine. Coating materials of these three different categories for targeting to the large intestine have been reviewed for example in Bansal et al. (Polim. Med. 2014, 44, 2, 109-118). Coating materials include, for example, poly vinyl acetate phthalate, cellulose acetate trimellitate, hydroxypropyl methylcellulose phthalate HP-50, hydroxypropyl methylcellulose phthalate HP-55, hydroxypropyl methylcellulose phthalate HP-55S, hydroxypropyl methylcellulose acetate succinate, cellulose acetate phthalate, acrylic acid copolymer, Eudragit L100-55, Eudragit L30D-55, Eudragit L-100, Eudragit L12.5, Eudragit S-100, Eudragit S12.5, Eudragit FS30D, hydroxyl propylethyl cellolose phthalate, PEG 6000, Ac-di-sol, Talc, hydroxy propyl methyl cellulose acetate succinate (HPMCAS), hydroxy ethyl cellulose, ethylcellulose, microcrystalline cellulose, hydroxy propyl methyl cellulose, chondroitin sulphate, pectin, guar gum, chitosan, lactose, maltose, cellobiose, inulin, cyclodextrin, lactulose, raffinose, stachyose, alginate, dextran, xantham gum, guar gum, starch, tragacanth, locust bean gum, cellulose, arabinogalactan, amylose and combinations thereof.

Solutions: In some instances, the formulation is distributed or packaged in a liquid form (e.g., suspension) for oral administration, for administration as an enema, or administration by instillation into a body cavity or lumen. Alternatively, formulations for non-injectable administration can be packaged as a solid, obtained, for example, by lyophilization of a suitable liquid formulation. The solid can be reconstituted with an appropriate carrier or diluent prior to administration.

Solutions and suspensions of the nanoparticles and/or microparticles can be prepared in water or another solvent or dispersing medium suitably mixed with one or more pharmaceutically acceptable excipients including, but not limited to, surfactants, dispersants, emulsifiers, pH modifying agents, and combination thereof.

The solution formulation is typically buffered to a pH of 3-8 for administration upon reconstitution. Suitable buffers are well known by those skilled in the art and some examples of useful buffers are acetate, borate, carbonate, citrate, and phosphate buffers.

Solutions, suspensions, or emulsions for administration may also contain one or more tonicity agents to adjust the isotonic range of the formulation. Suitable tonicity agents are well known in the art. Examples include glycerin, mannitol, sorbitol, sodium chloride, and other electrolytes.

Solutions, suspensions, or emulsions for administration may also contain one or more surfactants. Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or nanoparticles and/or microparticles.

Gelatin Capsules and Tablets: Tablets and inserts/suppositories can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations are prepared using pharmaceutically acceptable carriers including but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Optional pharmaceutically acceptable excipients include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar. The usual diluents include inert powdered substances such as starches, powdered cellulose, especially crystalline and microcrystalline cellulose, sugars such as fructose, mannitol and sucrose, grain flours, and similar edible powders. Typical diluents include, for example, various types of starch, lactose, mannitol, kaolin, calcium phosphate or sulfate, inorganic salts such as sodium chloride, and powdered sugar. Powdered cellulose derivatives are also useful.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin, and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

A lubricant can be used in a tablet formulation to prevent the tablet and punches from sticking in the die to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (POLYPLASDONE™ XL from GAF Chemical Corp.).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; vitamin E, tocopherol and its salts; sulfites such as sodium metabisulphite; cysteine and its derivatives; citric acid; propyl gallate, and butylated hydroxyanisole (BHA).

In some embodiments, the weight percent of the gel particles in the tablet or capsule formulations (with excipients) is between about 2% and about 80%, or between about 5% and about 70%, or between about 10% and about 60%. In some embodiments, the excipients include sodium starch glycolate (as a disintegrant) and mannitol (as a filler). In some embodiments, the weight percent of the gel particles in the tablet or capsule formulations (with excipients) is between about 2% and about 80%, or between about 5% and about 70%, or between about 10% and about 60%. In some embodiments, the excipients include sodium starch glycolate (as a disintegrant) and mannitol (as a filler).

Rectal Inserts or Suppositories: Rectal inserts or suppositories are typically formed by the same techniques as tablets, with additional excipient for comfort once inserted, such as increased amounts of inserts. The size and shape are selected based on the route of administration. These shapes, sizes, and excipients are well known to those in the pharmaceutical compounding art.

Enteric, Delayed or Pulsatile Release Formulations and Blended Formulations: A number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

Coatings can be applied to the particles, tablets, capsules, or inserts to modify release and to increase residence time at the site of delivery. The coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine from the clinical studies.

Coatings may be formed with a different ratio of water soluble polymer, water insoluble polymers and/or pH dependent polymers, with or without water insoluble/water soluble non-polymeric excipient, to produce the desired release profile. The coating is either performed on dosage form (matrix or simple) which includes, but are not limited to, tablets (compressed with or without coated beads), capsules (with or without coated beads), beads, particle compositions, and “ingredient as is” formulated as, but not limited to, suspension form or as a sprinkle dosage form.

Additionally, the coating material may contain conventional carriers such as plasticizers, pigments, colorants, glidants, stabilization agents, pore formers and surfactants.

The peptides embodied here, can be lyophilized, loaded onto microfibers which can be adsorbed onto microcrystalline cellulose beads (e.g., 60-250 μm mesh, or as large as 1,000 μm mesh) using a dry layering or suspension layering process. The microbeads are then coated by a fluidized bed coating process. Examples of coatings include pH responsive enteric coating, sustained released coating, and controlled release coating. In some embodiments, multi-layered coatings can be applied. The coated microbeads can be administered as a solid oral dosage form by loading them into a capsule or table. Alternatively, the coated microbeads can be suspended in water, buffer or other media and delivered as a liquid dosage form. Other buffering agents and excipients may be added to the liquid dosage form.

Enteric Coatings: The particles, tablets, capsules, or inserts may be coated to delay release to after the particles have passed through the acidic environment of the stomach. These materials are usually referred to as enteric coatings. For example, enteric polymers become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

Exemplary enteric polymers include polymethacrylates and derivatives thereof, such as ethyl methacrylate-methacrylic acid copolymer and those sold under the tradename EUDRAGIT™, naturally occurring cellulosic polymers (e.g., cellulose acetate succinate, cellulose acetate phthalate, hydroxy propyl methyl cellulose phthalate, and hydroxy propyl methyl cellulose acetate succinate) and other polysaccharides (e.g., sodium alignate, pectin, chitosan) or semi-synthetic or synthetic derivatives thereof, poly(2-vinylpyridine-co-styrene), polyvinyl acetate phthalate, shellac, fatty acids (e.g., stearic acid), waxes, plastics, and plant fibers.

Exemplary gastric resistant natural polymers include, but are not limited to, pectin and pectin-like polymers which typically consist mainly of galacturonic acid and galacturonic acid methyl ester units forming linear polysaccharide chains. Typically these polysaccharides are rich in galacturonic acid, rhamnose, arabinose and galactose, for example the polygalacturonans, rhamnogalacturonans and some arabinans, galactans and arabinogalactans. These are normally classified according to the degree of esterification. In high (methyl) ester (“HM”) pectin, a relatively high portion of the carboxyl groups occur as methyl esters, and the remaining carboxylic acid groups are in the form of the free acid or as its ammonium, potassium, calcium or sodium salt. Useful properties may vary with the degree of esterification and with the degree of polymerization.

Pectin, in which less than 50% of the carboxyl acid units occur as the methyl ester, is normally referred to as low (methyl) ester or LM-pectin. In general, low ester pectin is obtained from high ester pectin by treatment at mild acidic or alkaline conditions. Amidated pectin is obtained from high ester pectin when ammonia is used in the alkaline deesterification process. In this type of pectin some of the remaining carboxylic acid groups have been transformed into the acid amide. The useful properties of amidated pectin may vary with the proportion of ester and amide units and with the degree of polymerization.

Synthetic enteric polymers include, but are not limited to, acrylic acid polymers and copolymers, preferably formed from acrylic acid, methacrylic acid, methyl acrylate, ethyl acrylate, methyl methacrylate and/or ethyl methacrylate, and methacrylic resins that are commercially available under the tradename EUDRAGIT™ (Rohm Pharma; Westerstadt, Germany), including EUDRAGIT™ L30 D-55 and L100-55 (soluble at pH 5.5 and above), EUDRAGIT™ L-100 (soluble at pH 6.0 and above), EUDRAGIT™ S (soluble at pH 7.0 and above, as a result of a higher degree of esterification), and EUDRAGIT™ NE, RL and RS (water-insoluble polymers having different degrees of permeability and expandability).

The enteric coating is generally present in an amount less than about 10% by weight of the composition (e.g., gel particles, tablets, or capsules), preferably from about 2 to about 8% by weight of the composition.

The dosage units may be coated with the delayed release polymer coating using conventional techniques, e.g., using a conventional coating pan, an airless spray technique, or fluidized bed coating equipment (with or without a Wurster insert). See Pharmaceutical Dosage Forms: Tablets, Eds. Lieberman et al. (New York: Marcel Dekker, Inc., 1989), and Ansel et al., Pharmaceutical Dosage Forms and Drug Delivery Systems, 6th Ed. (Media, Pa.: Williams & Wilkins, 1995) for detailed information concerning materials, equipment and processes for preparing tablets and delayed release dosage forms.

Extended Release Drug/Particle Blends: One method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

The extended release formulations are generally prepared as diffusion or osmotic systems, for example, as described in “Remington—The Science and Practice of Pharmacy” (20th Ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, CARBOPOL™ 934, and polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion. The formulations with different drug release mechanisms can be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours, and similar edible powders. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders include substances such as starch, gelatin, and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose, and waxes can also serve as binders. A lubricant can be used in a tablet formulation to prevent the tablet and punches from sticking in the die. The lubricant is chosen from such slippery solids as talc, magnesium and calcium stearate, stearic acid, and hydrogenated vegetable oils.

Extended release tablets or inserts containing wax materials are generally prepared using methods known in the art such as a direct blend method, a congealing method, and an aqueous dispersion method. In a congealing method, the drug is mixed with a wax material and either spray-congealed or congealed and screened and processed.

Delayed Release Dosage Forms: Delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate, and carboxymethylcellulose sodium; acrylic acid polymers and copolymers, formed from acrylic acid, methacrylic acid, methyl acrylate, ethyl acrylate, methyl methacrylate and/or ethyl methacrylate, and other methacrylic resins that are commercially available under the tradename EUDRAGIT™ (Rohm Pharma; Westerstadt, Germany), including EUDRAGIT™ L30D-55 and L100-55 (soluble at pH 5.5 and above), EUDRAGIT™ L-100 (soluble at pH 6.0 and above), EUDRAGIT™ S (soluble at pH 7.0 and above, as a result of a higher degree of esterification), and EUDRAGIT™ NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The coating weights for particular coating materials may be readily determined by those skilled in the art by evaluating individual release profiles for tablets, beads and granules prepared with different quantities of various coating materials. It is the combination of materials, method and form of application that produce the desired release characteristics.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Pulsatile Release Formulations: By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design, e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In certain embodiments, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than three tablets, dosage forms housing more than three tablets can be utilized.

Alternatively, each dosage unit in the capsule may comprise a plurality of drug-containing beads, granules or particles. Drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. Drug-containing “granules” and “particles” comprise drug particles that may or may not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

In another embodiment, the individual dosage units are compacted in a single tablet, and may represent integral but discrete segments thereof (e.g., layers), or may be present as a simple admixture. For example, drug-containing beads, granules or particles with different drug release profiles (e.g., immediate and delayed release profiles) can be compressed together into a single tablet using conventional tableting means.

In a further alternative embodiment, a dosage form is provided that comprises an inner drug-containing core and at least one drug-containing layer surrounding the inner core. For dosage forms mimicking twice daily dosing, the dosage form has an outer layer that releases the active agents, e.g. peptide comprising the amino acid sequence, SEQ ID NO: 1, substantially immediately following entry into the intestine and an inner core having a polymeric-coating that preferably releases the active agent, e.g. peptide comprising the amino acid sequence, SEQ ID NO: 1, antibiotics, inflammatory agents, approximately 3 hours to less than 14 hours following ingestion of the dosage unit. For dosage forms mimicking three times daily dosing, the dosage form has an outer layer that releases the active agents, for example, within an hour following ingestion, an inner core that releases drug at least 5 hours to 18 hours following oral administration and a layer interposed between the inner core and outer layer that releases drug approximately 3 hours to 10 hours following ingestion of the dosage form. The inner core of the dosage form mimicking three times daily dosing may be formulated as compressed delayed release beads or granules.

Alternatively, for dosage forms mimicking three times daily dosing, the dosage form has an outer layer and an inner layer free of drug. The outer layer releases drug substantially within an hour following oral administration, and completely surrounds the inner layer. The inner layer surrounds both the second and third doses and preferably prevents release of these doses for approximately 3 hours to 10 hours following oral administration. Once released, the second dose is immediately available while the third dose is formulated as delayed release beads or granules such that release of the third dose is effected approximately 2 hours to 8 hours thereafter effectively resulting in release of the third dose at least 5 hours to approximately 18 hours following ingestion of the dosage form. The second and third doses may be formulated by admixing immediate release and delayed release beads, granules or particles and compressing the admixture to form a second and third dose-containing core followed by coating the core with a polymer coating to achieve the desired three times daily dosing profile.

Film-Forming Polymers for Coating Capsules: The film-forming composition can be used to prepare soft or hard shell gelatin capsules which can encapsulate a liquid or semi-solid fill material or a solid tablet (e.g., SOFTLET™) containing an active agent and one or more pharmaceutically acceptable excipients. Alternatively, the composition can be administered as a liquid with an active agent dissolved or dispersed in the composition. Exemplary film-forming natural polymers include, but are not limited to, gelatin and gelatin-like polymers. In certain embodiments, the film-forming natural polymer is gelatin. A number of other gelatin-like polymers are available commercially. The film-forming natural polymer is present in an amount from about 20 to about 40% by weight of the composition, or from about 25 to about 40% by weight of the composition.

The film-forming composition can be used to prepare soft or hard capsules using techniques well known in the art. For example, soft capsules are typically produced using a rotary die encapsulation process. Fill formulations are fed into the encapsulation machine by gravity. The capsule shell can contain one or more plasticizers selected from the group consisting of glycerin, sorbitol, sorbitans, maltitol, glycerol, polyethylene glycol, polyalcohols with 3 to 6 carbon atoms, citric acid, citric acid esters, triethyl citrate and combinations thereof. In addition to the plasticizer(s), the capsule shell can include other suitable shell additives such as opacifiers, colorants, humectants, preservatives, flavorings, and buffering salts and acids.

Opacifiers are used to opacify the capsule shell when the encapsulated active agents are light sensitive. Suitable opacifiers include titanium dioxide, zinc oxide, calcium carbonate, and combinations thereof. Colorants can be used for marketing and product identification/differentiation purposes. Suitable colorants include synthetic and natural dyes and combinations thereof.

Humectants can be used to suppress the water activity of the soft gel. Suitable humectants include glycerin and sorbitol, which are often components of the plasticizer composition. Due to the low water activity of dried, properly stored soft gels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

Mucoadhesive Particles: In general terms, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., van der Waals interactions) and stronger specific interactions, which include hydrogen bonds. The hydrophilic functional groups responsible for forming hydrogen bonds are the hydroxyl (—OH) and the carboxylic groups (—COOH).

Suitable polymers that can be used to form bioadhesive coatings include soluble and insoluble, biodegradable and non-biodegradable polymers. These can be hydrogels or thermoplastics, homopolymers, copolymers or blends, natural or synthetic. Two classes of polymers have appeared to show useful bioadhesive properties: hydrophilic polymers and hydrogels. In the large class of hydrophilic polymers, those containing carboxylic groups (e.g., poly(acrylic acid)) exhibit the best bioadhesive properties. In other studies, the most promising polymers were: sodium alginate, carboxymethylcellulose, hydroxymethylcellulose and methylcellulose. Some of these materials are water-soluble, while others are hydrogels.

Bioerodible polymers such as poly(lactide-co-glycolide), polyanhydrides, and polyorthoesters, whose carboxylic groups are exposed on the external surface as their smooth surface erodes, are excellent candidates for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, casein, gelatin, gluten, serum albumin, or collagen, polysaccharides, such as cellulose, dextrans, poly(hyaluronic acid), and polymers of acrylic and methacrylic esters and alginic acid. Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, and copolymers thereof. Synthetically modified natural polymers include alkyl celluloses, hydroxyalkyl celluloses, cellulose ethers, cellulose esters, and nitrocelluloses. The attachment of any positively charged ligand, such as polyethyleneimine or polylysine, may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge. Any ligand with a high binding affinity for mucin could also be covalently linked and be expected to influence the binding of microspheres to the gut. The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range would yield chains of 120 to 425 amino acid residues attached to the surface of the microspheres. The polyamino chains would increase bioadhesion by means of chain entanglement in mucin strands as well as by increased carboxylic charge.

### Routes of Administration

The formulations are administered orally, rectally or vaginally, or into any body lumen using instillation, an enema or insertion as of a suppository or insert. The formulation can be swallowed if administered in the form of a suspension, tablet or capsule. The formulation is administered by syringe, catheter, or instillation syringe if administered by instillation.

While in most cases administration is non-invasive, the formulation may be administered during surgery or minimally invasive procedures such as laparoscopy.

In certain embodiments, the pharmaceutical compositions are administered rectally. Rectal administration in context of the present disclosure means the introduction of the composition into gastrointestinal tract via the anus. In certain embodiments, the pharmaceutical compositions are administered in the form of an enema, a gel, a foam or a suppository.

For rectal administration as an enema, a gel or foam, the pharmaceutical compositions can include a hypotonic carrier. The hypotonic carrier will typically be a biocompatible carrier that preferably causes little to no signs of irritation when administered to human subjects. The carrier can be naturally occurring or non-naturally occurring including both synthetic and semi-synthetic carriers. Preferred carriers are sodium-based. Other solutions, including sugar-based (e.g. glucose, mannitol) solutions and various buffers (phosphate-buffers, tris-buffers, HEPES), may also be used.

When hypotonic solutions are applied to an epithelial surface, water flows out of the lumen, into cells and across the epithelium. This can cause swelling of the epithelial cells. In some cases, when the osmotic pressure difference is too large, the epithelial cells may burst, causing tissue irritation or disruption of the epithelial lining.

Advective transport of solutes is dominated by the bulk flow of a fluid, as in a solution passing through a filter. Since the colon absorbs water to dry the feces, fluid absorption by the colorectum can transport drugs advectively to the epithelium with great rapidity, much faster than by diffusion, and can move solutes through the unstirred layer of mucus adhering to the colonic epithelium. This distributes solutes to the entire colorectal surface, and if the formulation composition selectively improves tissue absorption rather than systemic absorption, minimizes systemic toxic side effects. The formulation for drug delivery markedly improves the uniformity of distribution of drugs over the epithelial surface.

An absorption-inducing (hypotonic) enema delivers drugs advectively to the colon epithelium by the bulk flow of water (advection) and is nontoxic. The absorption-inducing hypotonic enema formulations caused free drug to be transported rapidly to the epithelial surface, unimpeded by the unstirred mucus barrier coating the epithelium. A “hypotonic” solution refers to a solution that contains less impermeable solutes compared to the cytoplasm of the cell. Examples of hypotonic solutions include, but are not limited to, Tris[hydroxylmethyl]-aminomethane hydrochloride (Tris-HCl, 10-100 mM, pH. 6-8), (4-(2-hydroxyethyl)-1-piperazineethanesulfonic acid (HEPES, 10-100 mM, pH 6-8) and dilute solutions of PBS or normal saline (typically containing 0.9% NaCl).

The hypotonic carrier usually contains water as the major component. The hypotonic carrier can be water, although mixtures of water and a water-miscible organic solvent can also be used. Suitable water-miscible organic solvents include alcohols, such as ethanol, isopropanol; ketones, such as acetone; ethers, such as dioxane and esters such as ethyl acetate.

The hypotonic carrier can be water containing one or more tonicity modifying excipients. Sodium chloride is the excipient that is most frequently used to adjust tonicity of a solution. Other excipients used to adjust the tonicity of solutions include glucose, mannitol, glycerol, propylene glycol and sodium sulfate. Tonicity modifying excipients can include pharmaceutically acceptable salts such as sodium chloride, sodium sulfate, or potassium chloride. Other excipients used to adjust tonicity can include glucose, mannitol, glycerol, or propylene glycol.

The tonicity of a formulation varies for different cells and mucosal surfaces; it also depends on whether or not the cell or epithelium actively transports solutes and ions; e.g. it has been found that the isotonic point in the vagina for sodium-based solutions is about 300 mOsm/L, similar to the osmolarity of serum, but in the colorectum, it is significantly higher, about 450 mOsm/L (presumable because the colorectum actively transports sodium ions out of the lumen). In some embodiments the solution has a tonicity from 50 mOsm/L to 280 mOsm/L, from 100 mOsm/L to 280 mOsm/L, from 150 mOsm/L to 250 mOsm/L, from 200 mOsm/L to 250 mOsm/L, from 220 mOsm/L to 250 mOsm/L, from 220 mOsm/L to 260 mOsm/L, from 220 mOsm/L to 270 mOsm/L, or from 220 mOsm/L to 280 mOsm/L.

The hypotonic carrier can include one or more pharmaceutically acceptable acids, one or more pharmaceutically acceptable bases, or salts thereof. Pharmaceutically acceptable acids include hydrobromic, hydrochloric, and sulfuric acids, and organic acids, such as lactic acid, methanesulfonic acids, tartaric acids, and malic acids. Pharmaceutically acceptable bases include alkali metal (e.g. sodium or potassium) and alkali earth metal (e.g. calcium or magnesium) hydroxides and organic bases such as pharmaceutically acceptable amines. The hypotonic carrier can include pharmaceutically acceptable buffers such as citrate buffers or phosphate buffers.

In certain embodiments, the enema comprises at least one unit dose of the pharmaceutical compositions comprising the therapeutically effective amounts of the one or more antimicrobial peptides embodied herein, in the range of from about 0.1 mg to about 500 mg, or from about 1 mg to about 250 mg, or from about 2 mg to about 200 mg. In certain embodiments, the enemas are administered to the patient one to four times per day. In one embodiment, the enema is administered once daily. In one embodiment, the composition is administered twice daily. In another embodiment, the composition is administered three times per day. In another embodiment, the composition is administered three times per day.

Gel-forming compositions that are capable of forming uniform gel coatings on epithelial surfaces but do not gel under storage conditions can be used. The gel-forming compositions contain one or more gel-forming polymers in a hypotonic carrier, optionally containing one or more additional excipients and/or one or more therapeutic, prophylactic, or diagnostic agents.

### Dosages

The therapeutically effective dosage is readily determined from the known pharmacokinetics of the therapeutic, prophylactic or diagnostic agents, modified in view of the kinetics measured in vitro and in animal and human clinical trials, as is routinely done by those skilled in the art. The terms “sufficient” and “effective”, as used interchangeably herein, refer to an amount (e.g. mass, volume, dosage, concentration, and/or time period) needed to achieve one or more desired result(s) or alleviation of one or more symptoms of the disease or disorder.

### EXAMPLES

### Example 1: Induction of Colonic Sensitization in Neonatal Mice

Mouse pups at postnatal day 10 will receive a 0.2 ml enema infusion with 0.5% acetic acid or saline. The pup will be gently held by hand with head down. A poly-ethylene tubing (PE-10) connected to a 100 ul Hamilton syringe is insert through the rectum into the distal colon about 1.5 cm from the anus. 20 μl of 0.5% acetic acid or saline is infused slowly (about second). The pups are put back with dam and wean at 3 weeks old. The mice are allowed to grow into adulthood (8-12 weeks) when they are ready for test of hypersensitivity (increased pain to graded painful stimulus). These mice show no permanent damage (inflammation) to the colon in neonatal period or in adulthood but when these pups are allowed to grow into adult hood ˜80-90% of acetic acid sensitized rats exhibit hypersensitivity to the pain stimulus (colorectal distention, CRD).

### Example 2: Readouts for Pain

Visceromotor reflex (VMR) and Abdominal Withdrawal Reflex (AWR) response to colorectal distention (CRD) for assessing visceral pain sensitivity. The VMR is a reflex measured using an electromyographic (EMG) recording obtained from the external oblique muscle. First, a pair of electrodes is implanted into the external oblique muscle. 5-7 days later, a balloon is inserted into the colorectum. Then, 30-45 min later, the VMR response to CRD is measured by EMG.

Procedure of electrodes implantation: Under a cocktail of Ketamine/Xylazine (80:8 mg/kg body weight) or 1-2% isoflurane. adult mice undergo surgery to implant abdominal electrodes. The depth of anesthesia will be assessed by pinching the toes for a toe pinch response (negative) every 15 minutes during the surgical procedure. The abdominal area will be clipped and animal surgically prepped. All surgical procedure will be done aseptically. A small left lateral incision will be made in the lower abdominal area to expose the external oblique muscle. A sterilized multi-stranded, Teflon-insulated, 40-gauge stainless steel wires (Cooner Wire, Chatsworth, CA) is implanted into external oblique muscle. The wire is exposed about 0.5 cm at the end that is induced into the muscle by a 21 gauge needle. The wire is then fixed using suture. Two wires are inserted with toward different direction, so that they are not attached to each other. It is important that the wire is in the muscle but not into the abdomen lumen. The wires are then exteriorized at the dorsal neck area by subcutaneously. The skin will be closed with coated vicryl rapide suture and the rats will be rested visceral sensitivity responses a week later after rat recover from surgery. During the recovery period the animals will be placed on a clean absorbent surface in a clean cage and kept warm by placing a light on the cage until animals woken up.

Balloon insertion: A flexible balloon (made from 3 cm×3 cm square of polyethylene membrane) is attached to a Tygon catheter that is connected to a sphygmomanometer. Under mild sedation (2% isoflurane), the balloon is inserted into rat colorectum (about 2 cm) through anus and the tubing will be secured with a tape to the tail. Mouse will be placed in a mouse restrainer and allowed to adapt for 30-45 minutes before testing the VMR response to CRD by EMG.

EMG response to CRD: EMG is recorded from two externalized electrodes implanted in the external oblique muscle by using CED 1401 plus and Spike 2 software. A baseline of EMG is recorded for 20 second. Then the CRD is conducted with 15, 30, 50 or 70 mmHg for second followed by another 20 sec post-CRD recording. There are at least 2 minutes resting between the CRDs. The raw EMG signal is biphasic and is calculated to area under the curve in Spike 2. The data from each response are normalized to the baseline of each intensity of CRD.

Abdominal withdrawal response (AWR) to CRD is examined by visual observation of the behavioral responses to CRD. The observation is conducted by a blinded observer. On the day of the test, the balloon is inserted as above and 30-34 min later After CRD (20, 40, 60 or 80 mmHg) for 20 second and rest for 5 min, the behaviors of animal is graded with the AWR score as follows: 1=Normal behavior without response, 2=Contraction of abdominal muscles, 3=Lifting of abdominal wall and 4=Body arching and lifting of pelvic structures.

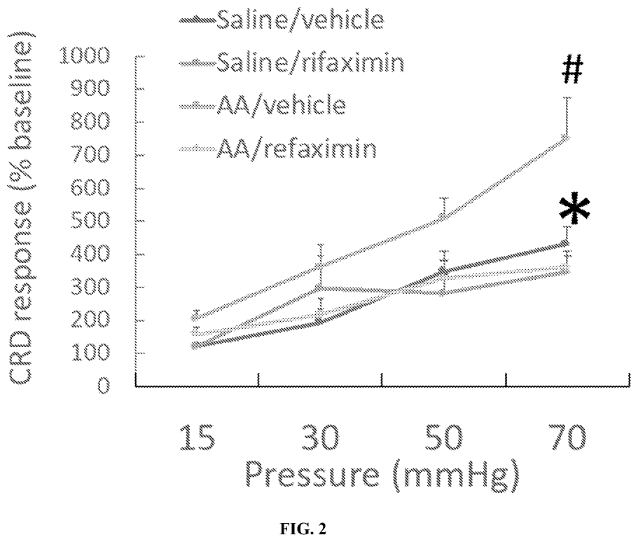

The results obtained demonstrate that rifaximin also attenuates pain behavior much in the same way as KSL-W (FIG. 2).

### Example 3: IBS Animal Model

Using a novel comprehensive, high-density DNA microarray (PhyloChip) a phylogenetic analysis of the microbial community of the large bowel was performed. The results revealed a significantly increased compositional difference in the microbial communities in “IBS” animals as compared with controls. Even more striking was the dramatic change in the ratio of Firmicutes relative to Bacteroidetes, where “IBS” animals were enriched more with Bacteroidetes and also contained a different composition of species within this phylum. The study also revealed differences at the level of bacterial families and species, incorporated herein by reference in its entirety (Nelson, T A et al. “PhyloChip microarray analysis reveals altered gastrointestinal microbial communities in a rat model of colonic hypersensitivity.” Neurogastroenterology and Motility. vol. 23.2 (2011): 169-77, e41-2. doi:10.1111/j.1365-2982.2010.01637.x).

These findings are consistent with multiple studies from humans with IBS showing alteration in the colonic microflora and have provided the basis for attempts to modulate the same with therapeutic intent. This includes fecal microbial transplants, recently shown to be successful (El-Salhy M, et al. Gut 2019;0:1-9. doi:10.1136/gutjnl-2019-319630), as well as pharmacological agents. One of the latter, rifaximin is a luminal antibiotic, approved by the FDA for treatment of IBS, and widely used in the community. The rationale herein, for using the KSL-W, an antimicrobial peptide that works against pathogenic bacteria but spares beneficial ones, is that it will reset the microbial balance in the colon to provide the same results (FIG. 1).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating irritable bowel syndrome (IBS), comprising: administering to a subject in need of such treatment, a pharmaceutical composition comprising a therapeutically effective amount of a peptide comprising at least 90% sequence identity to NH₂-Lys-Lys-Val-Val-Phe-Trp-Val-Lys-Phe-Lys-CO NH₂ (SEQ ID NO 1) or analogs thereof; thereby treating IBS.

2. The method of claim 1, wherein the peptide comprises SEQ ID NO: 1.

3. The method of claim 1, wherein the peptide is administered orally or via an enteral route.

4. The method of claim 1, wherein the peptide is administered via a rectal route.

5. The method of claim 1, further comprising administering one or more prebiotics, probiotics, antibiotics or a combination thereof.

6. The method of claim 5, wherein the antibiotic is administered that is rifaximin.

7. A method of treating a bowel disease comprising: administering to a subject in need of such treatment, a pharmaceutical composition comprising a therapeutically effective amount of NH₂-Lys-Lys-Val-Val-Phe-Trp-Val-Lys-Phe-Lys-CO NH₂ (SEQ ID NO 1) or analogs thereof; thereby treating bowel disease.

8. The method of claim 7, wherein the pharmaceutical composition is administered orally or via an enteral route.

9. The method of claim 8, wherein the pharmaceutical composition is administered orally.

10. The method of claim 8, wherein the pharmaceutical composition is administered orally by swallowing of the capsule or tablet.

11. The method of claim 8, wherein the pharmaceutical composition is administered via a rectal route.

12. The method of claim 11, wherein the formulation is administered rectally as a suppository, insert or enema.

13. The method of claim 1 wherein the pharmaceutical composition is formulated by one or more methods comprising: PEGylation, nanoencapsulation, low pH resistant capsules or as a solution for colonic irrigation.

14. The method of claim lany one of claim 1, further comprising administering one or more prebiotics, probiotics, antibiotics, anti-inflammatories or a combination thereof.

15. The method of claim 1, wherein a bowel disease comprises any one or more of: irritable bowel syndrome (IBS), diarrhea-predominant Irritable Bowel Syndrome (dIBS), inflammatory bowel disease (IBD), Crohn's disease, post-surgical pouchitis, diverticulitis, traveler's diarrhea, ulcerative colitis, enteritis, small intestinal bacterial overgrowth, chronic pancreatitis, pancreatic insufficiency, colitis, diverticular disease, colorectal cancer and/or hepatic encephalopathy.

16. A pharmaceutical composition comprising:
a) a therapeutically effective amount of NH₂-Lys-Lys-Val-Val-Phe-Trp-Val-Lys-Phe-Lys-CO NH₂ (SEQ ID NO or.
b) a therapeutically effective amount of one or more antimicrobial peptides comprising one or more of SEQ ID NOS: 1-4, one or more prebiotics, probiotics, antibiotics, anti-inflammatory agents or combinations thereof.

17. (canceled)

18. The pharmaceutical composition of claim 16, wherein the pharmaceutical composition comprises one or more antimicrobial peptides that are pegylated.

19. The pharmaceutical composition of claim 16, wherein the pharmaceutical composition comprises one or more antibiotics that comprise: penicillins, tetracyclines, cephalosporins, quinolones, lincomycins, macrolides, sulfonamides, glycopeptides, aminoglycosides, carbapenems or combinations thereof.

20. The pharmaceutical composition of claim 16, wherein the pharmaceutical composition comprises rifaximin.


SEQUENCE LISTING (4 sequences)

SEQ ID NO: 1 - protein, 10 aa, Artificial Sequence
  Synthesis of Antimicrobial Peptide KSL-W
KKVVFWVKFK

SEQ ID NO: 2 - protein, 10 aa, Artificial Sequence
  Synthesis of Antimicrobial Peptide KSL-M
  MOD_RES (6)..(6): METHYLATION
KKVVFKVKFK

SEQ ID NO: 3 - protein, 25 aa, Artificial Sequence
  Synthesis of Antimicrobial Peptide H1-2
DDDEEKFLRRIGRYGKKVVFWVKFK

SEQ ID NO: 4 - protein, 10 aa, Artificial Sequence
  Synthesis of Antimicrobial Peptides KSL
KKVVFKVKFK